(19) United States
(12) Patent Application Publication
(10) Pub. No.: US 2024/0053372 A1
(43) Pub. Date: Feb. 15, 2024

(54) PROCESS MANAGEMENT OF MEDIA PREPARATION

(71) Applicant: Merck Patent GmbH, Darmstadt (DE)

(72) Inventors: Stephane OLIVIER, Molsheim (FR); Gaetan BOUR, Molsheim (FR)

(73) Assignee: Merck Patent GmbH, Darmstadt (DE)

(21) Appl. No.: 18/268,423

(22) PCT Filed: Dec. 20, 2021
(86) PCT No.: PCT/EP2021/086695

§ 371 Date: Jun. 20, 2023

(30) Foreign Application Priority Data
Dec. 21, 2020 (EP) 20306631.1

(51) Int. Cl. G01N 35/10; G01N 35/00

(52) U.S. Cl. CPC G01N 35/1002; G01N 35/00732; G01N 2035/00752

(57) ABSTRACT

System for performing a media preparation process controlled by a computer (7) further comprising a Media Preparation Module (12), a media bag (2) for storing the media in an unprepared state (13), a Dispensing Module (9, 9a) for storing the media in a finally prepared state (17), and a water providing system which connects the Media Preparation Module (12) with the media bag (2) and the Dispensing Module (9, 9a) and provides them with temperature adjustable water using a water sterilization filter set (4) wherein, the system (6) additionally comprises an attachable machine readable data storage (3) which contains information about the current state and/or operating parameters of the system parts, which are read, processed and used by the computer (7) to operate the system (6) according to required conditions.

The hereby described invention discloses a method and a system for the process management of media preparation using a barcode or a RFID.

The invention deals with the technological area of medical care, food analysis or microbial testing but also can find applications with hydroponic plant growth when disposable products are used.

A culture media preparation system for food or pharmaceutical testing requires the use of different ingredients which not only need to be fully identified but that also impact directly the way they will have to be transformed by the system to reach the expected final regulation conformity in the specific laboratory application.

There are many different versions of culture media preparation systems known in the state of the art which differ according to their operating requirements. A culture media preparation system which is used to perform microbial testing on food samples requires different needs regarding its components, control parameters etc. than a culture media preparation system used for pharmaceutical matters.

It is very common to use systems for the first use case of food preparation, like small kitchen appliance, for instance the Thermomix. Those appliances come with a cooking guideline including step-by-step cooking instructions.

The customer can view any recipe directly on the device. Time, temperature and speed functions are all preset. The device at least partly handles the cooking and prompts the customer to add the next ingredient.

Examples for food or pharmaceutical culture media preparation systems are for instance so called RTU media in bags, boxes, or bottles. The Ready To Use media is then dispensed using an external pump. As an alternative to RTU media, are the large media bags containing dry media—(for example μPrep from LabM, Dry-Bags from Oxoid and MediaBag from VWR. This 20 Liter capacity irradiated bag is prefilled with Dehydrated Media which can be reconstituted with 20 liter of RO/deionized water flowing through a sterilization filter. The filter is connected/disconnected by a standard quick connector under an aseptic environment and can be connected to a dispensing pump or gravimetric diluter.

Also known are media preparators in form of stainless steel vessels for mixing and heat sterilization of powder media mixed rehydrated water followed by media cooling. They do not comprise a media dispensing function, but can be connected to a dispensing pump or gravimetric dilutors.

In a general approach it also possible to classify media preparation systems into a master-slave system approach, which does media preparation only and a dispenser system approach, which does media dispensing only. Those systems usually either:
include multiple programs that can be chosen in their instrument menu (UI)
integrate an alphanumeric keyboard and display to create custom programs
are connected to a computer for uploading and downloading programs or info
can be connected to a USB flash drive

BUT: none of those solutions:
allow instant and consumable related uploading protocol
Neither allow transferring process information to the consumable

In the existing solutions the user can access the identification of the ingredients and consumables. He is reading or scanning the corresponding labels to know the catalogue number, the lot number, the expiry date etc. But the user himself must actively introduce the working parameter for the media preparation and dispensing into his system. Knowing that the media preparation recipe and the media dispensing parameters can be very critical and fully dependent of the initial compound, any change of raw material or consumable should be considered when adjusting those parameters. There is risk of mistake if the parameters are not set up properly. This could result in a non-conformity with the needed regulation or could potentially generate a false positive or negative result, impacting directly the decision to release or not the product.

Moreover, in the current solutions the system cannot warrant that the parameter entered by the user are ok, for example it does not prevent the user from using an expired media, making a wrong dilution, and/or using a consumable above the maximum qualified limits. In the current systems changing media type also requires complex steps to assure that the working parameters are in line with the intended applications.

The task of this patent application is therefore to find and disclose a system and a corresponding method for media preparation which improves the usability, reliability and safety of media preparation independent from the users knowledge.

This task has been solved by a system for media preparation controlled by a computer further comprising a media preparation module, a media bag for storing the media in an unprepared state, a dispensing module for delivering the media in a finally prepared state, and a water providing system which connects the media preparation module with the media bag and the dispensing module and provides them with temperature adjustable water using a water sterilization filter set wherein, the system additionally comprises an attachable machine readable data storage which contains information about the current state and/or operating parameters of the system parts, which are read, processed and used by the computer to operate the system according to required conditions. What kind of media is prepared with the system is not specified. It could be any kind of solution, also for instance food. But the preferred use case according to the design of the system is preferably the preparing of pharmaceutical media like a nutrient solution or a culture medium for microbial testing of food or pharmaceutical samples. The readable data storage contains therefore information about the components like the filter or the media bag and are evaluated by the computer. To do so the computer is preferably provided with specifications about what kind of media, filter etc is allowed to be used with the system and by combining those specifications with the information from the readable data storage the computer can operate the system efficiently and automatically. In general the computer recognizes the consumables' characteristics from the data contained on the label, and then processes the data to drive the various modules to create the media stock according to the characteristics, like volume, concentration, dissolution time, lifetime, etc. Additionally the computer makes sure the consumables are authorized for this system.

Advantageous and therefore preferred further developments of the disclosed system comprise of this invention emerge from the associated subclaims and from the description and the associated drawings.

One of those preferred further developments of the disclosed system comprise that the adjustable machine readable data storage is an either an RFID label or a barcode, preferably a data matrix code according to the GS1 standard, which is attached to the media bag and/or the water sterilization filter set. Basically every kind of machine readable data storage is usable as long as it gives the computer access to required information. Most handy are barcodes because they can be easily attached anywhere by printing them on a label and attaching that label on a more or less flat surface. They could be also printed directly on that surface. Bar codes in form of two-dimensional data matrix codes additionally have the advantage that they can store more information than one-dimensional bar codes. When using the GS1 standard there should be no problems with compatibility. If bar codes are for a reason not suitable or desirable, RFIDs can be used as an alternative. They are superior in data capacity and are easier to read out automatically.

Another one of those preferred further developments of the disclosed system comprise that the water providing system is integrated into the media preparation module system and further comprises a water supply for the temperature adjustable water, a four-way-valve for connecting the media bag with the dispensing module and the water supply and a filter set between the water supply and the four-way-valve. The use of a T-shaped four-way-valve allows an easier direct controlling of the media preparation process by the computer. Four-way means that all three openings of the valve can be open at the same time. The water is directed from the water supply to the media bag via the for-way-valve where the media can be prepared. Then the prepared media, preferably in form of a solution or liquid can be transferred through the same pipes via the four-way-valve to the dispension module. This makes the whole media transport process much easier as if separate pipes and connections would have been used. Of course it has to be ensured that no contamination occurs due to the dual use of the same pipes. Actions like flushing the pipes with hot water or using sanitizers can support that.

Another one of those preferred further developments of the disclosed system comprise that the required conditions comprise specific set values for the operating parameters like the maximum water volume for the filter and/or media bag, the life time of filter and/or media bag, the designation for the media, the media volume, the dissolution time for the media and safety keys for ensuring the use of suitable system components and also comprise further specific set values for the system components like a global trade item number, a product catalogue number, a lot number, a serial number and an expiry date. Those are only some examples of values and parameters which may be checked. The computer reads those information and evaluates them regarding to specific prescribed values and thresholds and then operates the system accordingly. If for example the information shows that the life time of the used filter or media bag has run out over a specific maximum threshold the media preparation will be denied or cancelled.

Another one of those preferred further developments of the disclosed system comprises that the computer is integrated into the media preparation module in form of a microcontroller or industrial pc and provides a user interface to control and/or adjust the information about and set values for the current state and/or operating parameters. While it is of course possible to use a personal computer or notebook which is connected to the media preparation module to operate the system an embedded approach, by using an integrated microcontroller board or circuit which is responsible for reading out the data storage and processing the data accordingly is the preferred solution. Instead of a microcontroller also an industrial pc can be used if it is possible to integrate it into the media preparation module. This microcontroller is also connected to and preferably controls the user interface, provided by the system, which allows the user to enter and retrieve commands and information.

Another one of those preferred further developments of the disclosed system comprises that in case of using a barcode as attachable machine readable data storage the system additionally comprises a barcode reader device which is connected to the computer for data transmission. If a barcode is used as a machine readable data storage there is a need for an external barcode reader which has a visible access to the surface of the barcode to read it. This could be either a standalone barcode reader device which is connected to the computer by wire or also using a wireless standard or it could be a mobile phone with a camera. This mobile phone could interpret the barcode directly via a suitable app and send the plain information to the computer or just send the scanned image to the computer via a data connection where the computer interprets it on its own. The mobile phone would be preferably connected to the computer by using a wireless connection like Bluetooth, wifi, etc.

Another one of those preferred further developments of the disclosed system comprises that in case of using an RFID label as attachable machine readable data storage the system additionally comprises a RFID reader device which reads and if necessary powers the RFID labels and is integrated into the media preparation module and connected to the computer for data transmission. When using RFID labels there is the advantage that no visible access from the RFID reader to the RFID label itself is necessary—like when using a barcode. That means the RFID reader can be integrated into the media preparation module resulting in much handier and useful solution as if an additional external device would be used. The RFID reader provides a data interface and connection to the computer so the latter has direct access to the read out information from the RFID labels. The RFID reader could also furthermore be integrated in the computer itself, especially if the computer is integrated in the media preparation module e.g. in form of a microcontroller board or circuit. It does not matter for the invention if the RFID is an active RFID with its own power supply or is powered passively by the RFID reader. In the last case the RFID reader needs to be able to power the RFID and therefore needs probably a more powerful power supply for itself. It is also possible to interconnected several systems and share the information about the usage information about the consumables using unique identifier coded in the label.

A further preferred embodiment of the disclosed system is that it comprises an external web tool is used to generate, preferable two-dimensional, barcodes which are read by the user via an web app and used to configure the computer. Use case for this web-app is that the lab manager can create a set of barcodes containing the dispensing information, like temperature, flow, concentration, etc, on a suitable web tool and the user in the lab only has to scan the barcodes with the app, in this case running on a mobile phone or similar, and doesn't need to enter the individual parameters by hand in the instrument anymore, risking input errors.

Another solution to the task is additionally a method for a computer controlled media preparation process using a system as previously described, comprising the steps of reading the attached machine readable data storage to acquire the required state and/or operating parameters of the system parts, adjusting the system settings according to the acquired state and/or operating parameters and to fix set values via the computer, transfer of heated water to the media bag using the computer controlled water providing system, dissolving dehydrated material in the media bag with the heated water until a first concentration level is reached, mix of filtered, heated water from the water supply and the filter with the dissolved material in the first concentration level from the media bag in the four-way-valve until a final concentration level for the media is reached and filling the dispensing module with a required amount of the finally prepared media. The dehydrated material usually consists of a kind of powder, but also any other suitable pharmaceutical form is possible. After the dissolving of the powder, the user can enter new parameters, like final media parameters, through barcode reading, e.g. generated with the web app, RFID, etc and modify them with the key pad, and store them in the internal database. Depending on the use case and samples to be processed, the user may need to change the final media parameters several times during the dispensing process, until the media bag is empty. Core part of the invented method is that the computer executes the process dependent on and optimized for the acquired information with preferred operating parameters from the machine readable data storage. When the finally prepared media is collected in the dispensing module, preferably in an removable bag or the like, it can be removed by the user for further applications.

Another one of those preferred further developments of the disclosed method comprise that the computer as part of adjusting the system settings checks if the acquired state and/or operating parameters of the system parts are matching their required set values and in case of critical exceedance performs actions to stop or adapt the media preparation process. That means if the specific required set values are not matching the acquired information from the machine readable data storage the computer automatically takes care that the media preparation process is not executed under wrong conditions to support the human user. As the whole preparation process is computer controlled there are many possibilities for the computer according to its program to adapt or even stop the media preparation process if necessary.

Another one of those preferred further developments of the disclosed method comprise that the actions to stop or adapt the media preparation process includes rejecting a recognized unsuitable media bag or filter, rejecting a filter or media bag if maximum allowed amount of heated water or media stock has been used, if there was an issue during the media bag filling process, and thus risk of wrong concentration in the bag, adaption of used heated water volume, prevent the delivery by the dispensing module if dissolving has not been reached, adjust a wrongly set water flow rate and/or temperature. Those are the most obvious options to adapt or stop the preparation process but the actions are not limited to them. Every action which helps to prevent executing a faulty media preparation process is included.

Another one of those preferred further developments of the disclosed method comprise that additionally a maintenance and/or a sanitization process is performed, supported by the computer by reading, processing and using further information from the attachable machine readable data storage which is related to that maintenance and/or a sanitization process. To execute a sanitization process it is e.g. possible to use hot water from the water supply or specific sanitizing solutions to sanitize or disinfect the used water and solution pipes. Maintenance actions could include for instance the exchange of the filter.

Another one of those preferred further developments of the disclosed method comprise that the further information about the sanitization process includes the number of sanitizing cycles, the volume of sanitizer liquid, the heated water or sanitizing solution flow during sanitization, the waiting time between each sanitization cycle, the number of rinsing cycle, the volume of rinsing liquid, the water flow during rinsing and the waiting time between each rinsing cycle. Also here those actions are only peculiar preferred examples but further actions are included as well.

Another one of those preferred further developments of the disclosed method comprise that a RFID with read and write capability is used and the computer is reading, processing and using not only the initial values of the information but recording in the RFID also all changed values which are impacting the media preparation process, like the remaining volume of water that can flow in the filter before to be rejected, the remaining number of connections for the filter, an information that the media bag has been reconstituted or not, the date and time of that reconstitution, the remaining volume of a reconstituted media in the bag, the estimated temperature of the bag, and the remaining time before end of use for the media bag. In this case the RFID reader needs to be capable of writing data to the RFID. The computer needs to be enabled to write data to the RFID as well, meaning that the data interface for data transfer between computer and RFID reader needs to support that feature.

A further solution to the task is also a software product performing one of the disclosed methods on the computer of the disclosed system. The software product includes especially the program running on the control computer for the system. But also any possible GUI or control software for the barcode or RFID reader including their data interfaces, are part of that software.

The system, method and software product according to the invention and functionally advantageous developments of those are described in more detail below with reference to the associated drawings using at least one preferred exemplary embodiment. In the drawings, elements that correspond to one another are provided with the same reference numerals.

The drawings show:

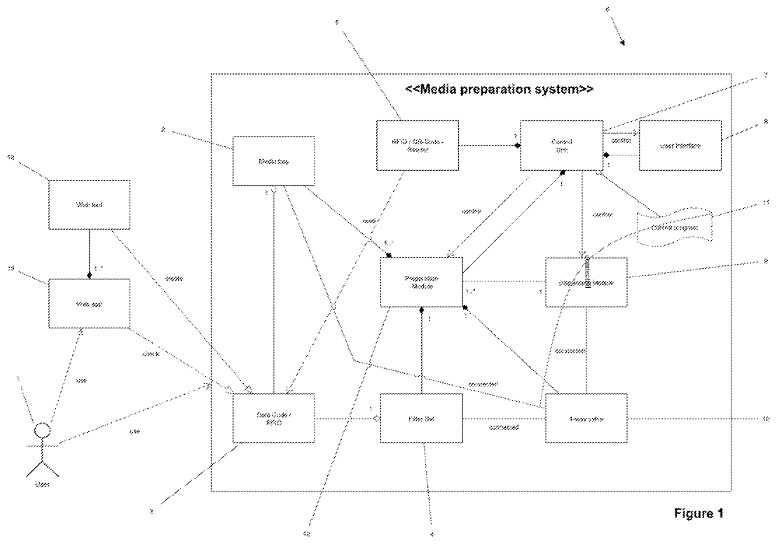

FIG. 1: an schematic overview about the components of the system for media preparation

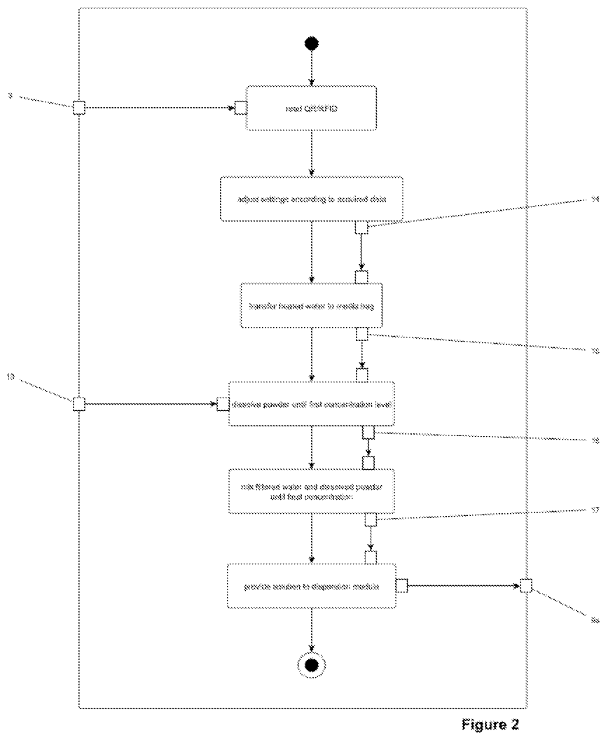

FIG. 2: a schematic process of the invented method for media preparation

The proposed invention follows several aims. Crucial points are e.g. to:
secure the media preparation process
secure the media dispensing process
prevent the use of any expired consumable
prevent dispensing from a media 17 that has not been prepared properly like wrong concentration, wrong temperature
prevent dispensing from a media 16 before it has been fully reconstituted
preventing dispensing from a reconstituted media after its qualified usage time
control the remaining volume and therefor the number of remaining tests that can be extracted and dispense from a reconstituted media
allow the manufacturing of a panel of evolutive critical media (batch to batch dependent) that can be recognize by the system 6 without the needs of updating the control program 11 (software) of the instrument
allow instant upload of very specific operating procedure without any risks
Use simple hardware with limited user interface which does not require complex software 11 and keypad on the hardware to interact with the user 1

The invented system 6 in a preferred embodiment is shown in a schematic overview in FIG. 1. Main parts of that system are a Media Preparation Module 12 and a Dispensing Module 9, 9a. The system 6 has the capability to store information and to run the media preparation process. To do so it comprises a Control Unit 7 which runs the control program 11 and is a kind of computer. Technically every computer which is able to run the control program 11 and has a control and data interface to at least the Preparation Module 12 can be used. In a preferred embodiment a microcontroller circuit is used which is integrated into the Preparation Module 12. The system 6 also comprises a user interface 8 which consists in a preferred embodiment of a display for showing information to the user 1 and a control pad which allows the user 1 to input commands and/or data within the limits the control program 11 allows. The computer 7 then collects data from Machine readable data storage 3 which is either a barcode or a RFID tag/label. In case of a barcode the preferred embodiment is a two-dimensional data matrix code, like a QR Code or GS1 datamatrix. Those data matrix code or the RFID tag or label are attached to either a media bag 2 which contains the media consumable set 13 and/or to a water sterilization filter set 4 which is integrated in the water supply of the system 6 comprising of a supply for heated water 15 with the temperature adjustable by the computer 7, and a pipe system with at least one pipe to transport the heated water 15 and/or later the prepared media solution 16, 17. The barcode is in general scanned only after the system is set up with the media bag. Another component of the system 6 is a four-way-valve 10 which is integrated in the water supply and allows to transport heated water 15 through the same pipe system from the water supply to the media bag 2 with the media powder 13 and then, as the media solution 16, 17 back to the valve 10 and then to the Dispensing Module 9, 9a.

With that system 6 the media preparation process can be executed in a preferred way. FIG. 2 shows a preferred example embodiment of the invented method which is performed on the invented system 6. To perform the media preparation process the Media Preparation Module 12, controlled by the computer 7, is pumping heated water (WFI—water for injection or Deionised water, or any kind of water suitable for the application) 15 at a specific flow and temperature through the pipes and the filter set 4. There it passes the four-way-valve 10 which ports are connected as follows:
one port is connected to the media bag 2 containing the media powder 13 or an already concentrated solution
the second port is connected to the heated water supply
the third port is connected to a Dispensing outlet, in this case the Dispensing Module 9, 9a

In a first step the computer 7 reads the barcode or the RFID label with help of its barcode or RFID reader 5. If it is an barcode the help of a human user 1 to point the barcode reader to the surface of the barcode is necessary. As barcode reader a specific device can be used in the preferred embodiment which has a data connection, wired or wireless, to the computer 7. In a further preferred embodiment a mobile phone with a camera is used. Here the barcode is read through a web app 18 and the mobile phone connects either direct to the computer, e.g. via Bluetooth or wifi, etc. or through intranet or internet network with local wifi network or mobile phone network.

If a RFID is used it can be read by its reader device 5 automatically when positioned near enough. To ensure that the RFID reader 5 is integrated in the Preparation Module 12 and directly connected to the computer 7 for the data transfer. With the specific data read out from the barcode or RFID the computer 7 then checks the current system settings 14 and adapts the media preparation process according to its specific needs.

The next step is then to transfer the heated water to the media bag 2 in order to dissolve the media powder 13 or solution to a first concentration level 16 (bulk concentrate). In a second step via the four-way-valve 10 the filtered water and the bulk concentrate 16 of the media bag 2 are mixed in order to dispense an expected volume of diluted media at the final concentration—the dispense concentration 17, at the defined temperature and flow rate. When the ready prepared media 17 has arrived in the Dispensing Module 9, 9a in the wanted quantity the process is over and the prepared media 17 can be taken by the user for further use. Eventually additional actions like cleaning, sanitizing or even maintaining the system 6 are performed afterwards.

To enable the computer 7 to check the current system settings 14 and adapts the media preparation the barcode or RFID needs to provide specific information. Those parameters are for instance but not limited to:

In general:
The Global Trade Item number (GTIN)
The product catalogue number
The lot number
The serial number
The expiry date

But also several more specific custom parameters are provided:
For the Filter set 4:
The Designation
The maximum of water volume that can be filtered through the unit to warranty the performances
the Filter life time, e.g. the maximum number of day after first use of the filter 4
the number of maximum connections of that filter 4 with different media bags 2 set to limit contamination risks
Safety key to avoid counterfeiting copy
For the Media bag 2:
The Designation
The bag life time, like the maximal time between reconstitution and full dispensing
The water volume, e.g. the volume of water to be added in the media bag 2 to get an initial volume of reconstituted media
The bag volume, .e.g. the total volume of media available after reconstitution with water
The media concentration, e.g. the initial media concentration after reconstitution with water
The dissolution time->the waiting time to warranty full dissolution before starting dispensing
Safety key to avoid counterfeiting copy That information collected by the computer 7 will warrant that:
1. The scanned filter 4 is:
   a suitable filter->a non-recognized filter set 4 will be rejected
   a filter set 4 with an acceptable shelf life->an expired filter 4 will be rejected
   inside the limits of day of usage-> if not it will be rejected
   inside the maximal cumulative volume of water that can be filtered-> if over that volume the filter 4 will be rejected
   connected to less than x media bags 2->otherwise it is not accepted to be use with more than x bag 2
2. The Media Bag set is:
   a suitable media bag 2 with an acceptable shelf life->an expired bag 2 will be rejected
   reconstituted with the right volume of water->the computer 7 will ensure to fill it with the expected controlled volume
   containing after reconstitution and dilution the right volume of the specified concentrated media 16, 17->will be the initial known condition before dispensing
   only possible to be dispense after full dissolution time->otherwise computer 7 will prevent dispensing undissolved media 13
   inside the limits of day of usage-> if out, the media bag 2 will be rejected
   containing enough remaining volume to perform the expected dispensing conditions->otherwise it is prevented from dispensing a volume that is not present in the bag 2

To use those information the computer 7 uses specific set values which are then compared with the new information he gets from reading the barcode and/or RFID. Those set values can be adapted by the user 1. Therefore he can use either the control panel and change them manually or uses the data matrix code or RFID itself. For the data matrix code a standard operating procedure (SOP) is applied. The data matrix code contains then, for example, the following information:
The volume of media 13 to be dispensed
The final expected concentration of that dispense media solution 17
The flow rate that is required
The temperature of the dispensed media 16, 17
The user priority according to flow or temperature->Priority to temperature means the flow will be adjusted to warranty the targeted temperature; Priority to flow means the wanted temperature may not be reached to warranty the expecting dispensing time or flow

The data matrix code is generated by the, or a different user 1 via a suitable web application tool 19

Also several types of barcode standards can be used to store and exchange the multiple parameters. In one preferred embodiment the GS1 standard data matrix (two dimensional barcodes) which is broadly used in healthcare, medical and pharmaceutical market. The GS1 standard is a unique and unambiguous identification system. It brings reading safety by using data redundancy using Reed-Solomon error correction to help correct for partially damaged symbols. It also allows having hx and variable data, including standardized and custom variable.

If needed the data matrix code can advantageously be replaced by a RFID label which has either read only or read-and-write-ability. In case of using a read-and-write-tag the user 1 has the possibility to store in the consumables like the filter set 4 and the media bag 2, not only the initial values but also all changes that have been impacting the process. That means the system 6 is able to record:
1. In the RFID of the Filter set consumable 4:
   The remaining volume of water 15 that can flow in the filter before it is rejected
   The remaining number of connections
   The date of 1^{st} use
2. In the RFID of the media bag consumable 2:
   The information that the bag 2 has been reconstituted or not
   The date and time of reconstitution
   The remaining volume of reconstituted media in the media bag 2
   The remaining time before end of use
   The indentification of the user who has prepared the solution

This has the additional advantage that data about changed parameters does not need to be recorded and tracked by the hardware of the system 6 or the user 1 itself but is part of the consumable (media bag 2 or filter set 4) which can be moved from one instrument to the other without any recognition problem. Also e.g. if a user logged in or scan an ID tag on the instrument, be it a RFID or barcode, and in addition to date/time of reconstitution, also the identifier of the operator who has reconstituted the media is therefor provided.

Further operating procedures which can additionally be programmed and performed by the system 6 and which are using data matrix codes or RFID labels as well are for example for an instrument sanitization process or a process for maintenance purposes. A sanitization process for example could require the following parameters stored in the code or RFID:
Number of sanitizing cycle
Volume of sanitizer liquid
Flow during sanitization
Waiting time between each sanitization cycle, like the contact time between internal surfaces of the fluidic path and sanitizing solution
Number of rinsing cycles
Volume of rinsing liquid
Flow during rinsing
Waiting time between each rinsing cycle

### LIST OF REFERENCES

1 User
2 Media bag
3 Machine readable data storage
4 Filter set
5 Barcode/RFID reader device
6 Media preparation system
7 Control unit/Computer
8 User interface
9 Dispensing Module
9a Dispensing Module with ready prepared media
10 Four-way-valve
11 Control program
12 Preparation Module
13 Media in powder form
14 System settings
15 Heated water (WFI)
16 Media solution in first concentration level
17 Media solution in final concentration level
18 Web tool
19 Portable web app

1. System for media preparation (6) controlled by a computer (7) further comprising a Media Preparation Module (12), a media bag (2) for storing the media in an unprepared state (13), a Dispensing Module (9, 9a) for delivering and/or storing the media in a finally prepared state (17), and a water providing system which connects the Media Preparation Module (12) with the media bag (2) and the Dispensing Module (9, 9a) and provides them with temperature adjustable water using a water sterilization filter set (4)
wherein,
the system (6) additionally comprises an attachable machine readable data storage (3) which contains information about the current state and/or operating parameters of the system parts, which are read, processed and used by the computer (7) to operate the system (6) according to required conditions.

2. System according to claim 1,
wherein
the adjustable machine readable data storage (3) is an either an RFID label or a barcode, preferably a data matrix code according to the GS1 standard, which is attached to the media bag (2) and/or the water sterilization filter set.

3. System according to claim 1,
wherein
the water providing system is integrated into the Media Preparation Module (12) and further comprises a water supply for the temperature adjustable water, a four-way-valve (10) for connecting the media bag (2) with the Dispensing Module (9, 9a) and the water supply and the filter set (4) between the water supply and the four-way-valve (10).

4. System according to claim 1,
wherein
the required conditions comprise specific set values for the operating parameters like the maximum water volume for the filter set (4) and/or media bag (2), the life time of filter set (4) and/or media bag (2), the designation for the media (13, 16, 17), the media volume, the dissolution time for the media (13) and safety keys for ensuring the use of suitable system components and also comprise further specific set values for the system components like a global trade item number, a product catalogue number, a lot number, a serial number and an expiry date.

5. System according to claim 1,
wherein
the computer (7) is integrated into the Media Preparation Module (12) in form of a microcontroller or industrial pc and provides a user interface (8) to control and/or adjust the information about and set values for the current state and/or operating parameters.

6. System according to claim 1,
wherein
in case of using a barcode as attachable machine readable data storage (3) the system (6) additionally comprises a barcode reader device (5) which is connected to the computer (7) for data transmission.

7. System according to claim 1,
wherein
in case of using an RFID label as attachable machine readable data storage (3) the system additionally comprises a RFID reader (5) device which reads and if necessary powers the RFID labels and is integrated into the Media Preparation Module (12) and connected to the computer (7) for data transmission.

8. System according to claim 1,
wherein
an external web tool (18) is used to generate, preferable two-dimensional, barcodes which are read by the user (1) via an web app (19) and used to configure the computer (7).

9. Method for a computer (7) controlled media preparation process using a system (6) according to claim 1, comprising the following steps:
Read the attached machine readable data storage (3), preferably a RFID or barcode, to acquire the required state and/or operating parameters of the system parts
Adjust the system settings (14) according to the acquired state and/or operating parameters and to fix set values via the computer (7)
Transfer heated water (15) to the media bag (2) using the computer (7) controlled water providing system
Dissolve dehydrated media material (13) in the media bag (2) with the heated water (15) until a first concentration level (16) is reached
Mix filtered, heated water from the water supply and the filter set (4) with the dissolved material in the first concentration level (16) from the media bag (2) in the four-way-valve (10) until a final concentration level (17) for the media is reached
Fill the Dispensing Module with a required amount of the finally prepared media (17)

10. Method according to claim 9
wherein,
the computer (7) as part of adjusting the system settings (14) checks if the acquired state and/or operating parameters of the system parts are matching their required set values and in case of critical exceedance performs actions to stop or adapt the media preparation process.

11. Method according to one claim 10,
wherein,
the actions to stop or adapt the media preparation process includes rejecting a recognized unsuitable media bag (2) or filter set (4), rejecting a filter set (4) or media bag (2) if maximum allowed amount of heated water (15) or media quantity has been used, correction of used heated water volume, prevent the filling of the Dispensing Module (9, 9a) if dissolving has not been reached, adjust a wrongly set water flow rate and/or temperature.

12. Method according to claim 9,
wherein,
additionally a maintenance and/or a sanitization process is performed, supported by the computer (7) by reading, processing and using further information from the attachable machine readable data storage (3) which is related to that maintenance and/or a sanitization process.

13. Method according to claim 12,
wherein,
the further information about the sanitization process includes the number of sanitizing cycles, the volume of sanitizer liquid, the heated water flow during sanitization, the waiting time between each sanitization cycle, the number of rinsing cycle, the volume of rinsing liquid, the heated water flow during rinsing and the waiting time between each rinsing cycle.

14. Method according to claim 9,
wherein,
a RFID with read and write capability is used and the computer (7) is reading, processing and using not only the initial values of the information but recording in the RFID also all changed values which are impacting the media preparation process, like the remaining volume of water that can flow in the filter set (4) before to be rejected, the remaining number of connections for the filter, an information that the media bag (2) has been reconstituted or not, the date and time of that reconstitution, the remaining volume of a reconstituted media in the media bag (2), and the remaining time before end of use for the media bag (2).

15. Software product (11) performing the method according to claim 9.
